(19) United States
(12) Patent Application Publication
(10) Pub. No.: US 2015/0208905 A1
(43) Pub. Date: Jul. 30, 2015

(54) ENDOSCOPE CABLE

(71) Applicant: Hitachi Metals, Ltd., Tokyo (JP)

(72) Inventors: Detian HUANG, Hitachi (JP); Takanobu WATANABE, Hitachi (JP); Kimika KUDO, Kitaibaraki (JP)

(21) Appl. No.: 14/308,159

(22) Filed: Jun. 18, 2014

(30) Foreign Application Priority Data
Jan. 24, 2014 (JP) 2014-011389

(51) Int. Cl. A61B 1/00; H01B 11/18; H01B 7/04; H01B 3/30; H01B 3/48

(52) U.S. Cl. CPC A61B 1/00114; H01B 3/302; H01B 3/48; H01B 7/04; H01B 11/1895

(57) ABSTRACT

An endoscope cable includes a hollow cylindrical flexible endoscope tube, and a woven cable helically arranged inside the endoscope tube.

The present application is based on Japanese patent application No. 2014-011389 filed on Jan. 24, 2014, the entire contents of which are incorporated herein by reference.

### BACKGROUND OF THE INVENTION

1. Field of the Invention

This invention relates principally to an endoscope cable used in an endoscopic tester with a CCD (Charge Coupled Device) image sensor.

2. Description of the Related Art

As shown in FIG. 4, an endoscopic tester with a CCD image sensor used for endoscopic testing is equipped with an endoscope cable 300 having accommodated inside a hollow cylindrical flexible endoscope tube 301 an electric cable 302 to electrically connect together the device body and the CCD image sensor, and provides electric power from the device body to the CCD image sensor, a light guide 303, an air/water supply channel 304, a suction channel 305, a treatment tool channel 306, etc. used in body observation or treatment.

Of these, as the electric cable 302, a multiconductor cable is known that includes a signal wire bundle formed by stranding a plurality of signal wires together, a presser wrap wrapped around the signal wire bundle, a shield conductor wrapped around the presser wrap, and a cable jacket provided around the shield conductor. Refer to e.g. JP-A-2007-188738, for example.

Refer to e.g. JP-A-2007-188738, JP-A-2009-170235, and JP-A-2006-286299.

### SUMMARY OF THE INVENTION

Now, when endoscopic testing is performed, due to need to insert the endoscope cable 300 from a patient's mouth or the like into body, the patient is likely to suffer some pain resulting from endoscopic testing. To mitigate the pain suffered by the patient receives as much as possible at this point, a diametrical reduction of the endoscope cable 300 has been demanded. Therefore, a diametrical reduction or space saving of the electric cable 302 has been required.

To achieve the diametrical reduction of the electric cable 302, it is conceivable to diametrically reduce the plurality of constituent signal wires of the multiconductor cable, but electrical characteristics and amount of electric power provided lower resulting from the diametrical reduction. Also, terminal workability tends to worsen, and wire breaking during handling tends to occur.

Also, to achieve the space saving of the electric cable 302, it is conceivable to adopt a flexible flat cable in place of the multiconductor cable as the electric cable 302, but in an attempt to make sufficient the flexibility of the flexible flat cable, no electromagnetic shielding, which lowers the flexibility thereof, can be applied thereto. Therefore, no requirements for electrical characteristics of the electric cable 302 can be satisfied.

Further, although the space saving of the electric cable 302 can be achieved by adopting a multiconductor cable having longitudinally arranged and accommodated therein a plurality of plain weave units comprising a plurality of electric wires plain woven with a weft, this multiconductor cable is not excellent in bending resistance and twisting resistance because the flexibility of the endoscope cable 300 depends on the flexibility of the plurality of electric wires due to the longitudinal arrangement of the plurality of plain weave units.

Accordingly, it is an object of the present invention to provide an endoscope cable, which is capable of enhancing bending resistance and twisting resistance, while achieving a further diametrical reduction as compared with a conventional diameter.
(1) According to an embodiment of the invention, an endoscope cable comprises:
   a hollow cylindrical flexible endoscope tube; and
   a woven cable helically arranged inside the endoscope tube.

In the embodiment, the following modifications and changes can be made.
(i) The woven cable includes a plurality of electric wires arranged side by side, and a fiber member woven in such a manner as to sew between the plurality of the electric wires and in a side by side arrangement direction of the plurality of the electric wires, and
the fiber member comprises a polyurethane elastic fiber.
(ii) The fiber member is being elongated by weaving in such a manner as to sew between the plurality of the electric wires.
(iii) The fiber member comprises a monofilament.
(iv) The woven cable stretches or compresses in a side by side arrangement direction of a plurality of electric wires thereof
(v) The electric wire is 0.23 mm or less in outer diameter, and is 0.25 mm or less in wiring pitch.

### (Points of the Invention)

The present invention can provide the endoscope cable, which is capable of enhancing bending resistance and twisting resistance, while achieving a further diametrical reduction as compared with a conventional diameter.

### BRIEF DESCRIPTION OF THE DRAWINGS

The preferred embodiments according to the invention will be explained below referring to the drawings, wherein:

FIG. 1 is a cross sectional view showing an endoscope cable in an embodiment according to the present invention;

FIG. 2 is a perspective view showing the endoscope cable of FIG. 1;

FIG. 3A is a cross sectional view of an electrically insulated wire;

FIG. 3B is a cross sectional view of a coaxial wire;

FIG. 3C is a cross sectional view of another coaxial wire; and

FIG. 4 is a cross sectional view showing a conventional endoscope cable.

### DETAILED DESCRIPTION OF THE EMBODIMENTS

Below is described an embodiment according to the invention, in conjunction with the accompanying drawings.

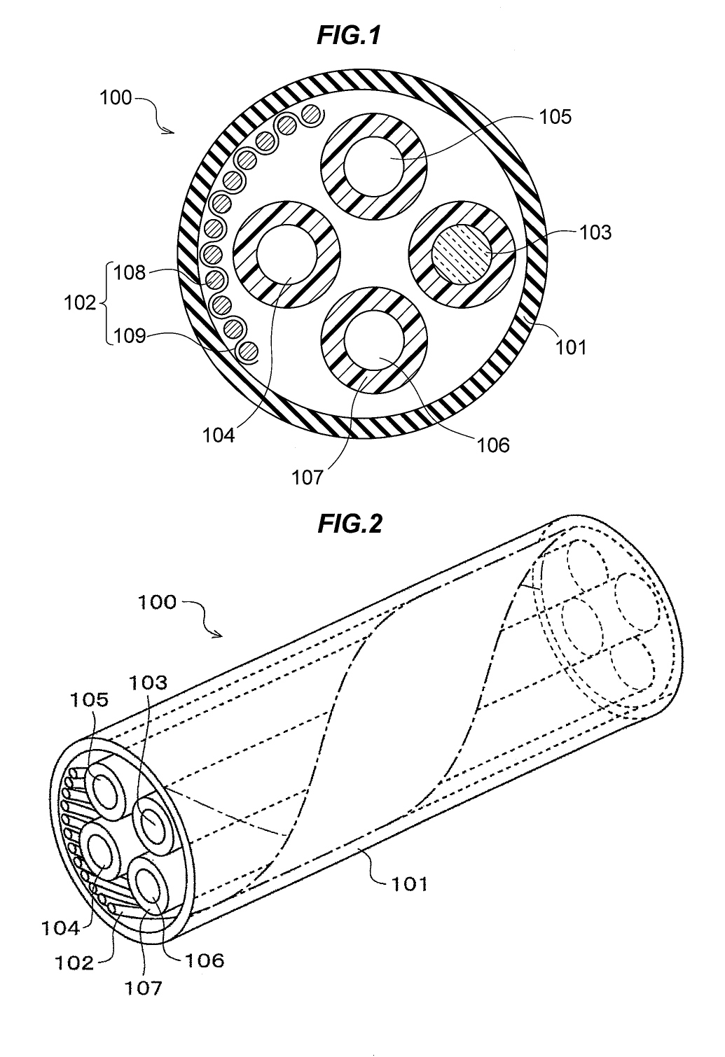

As shown in FIG. 1, an endoscope cable 100 in the present embodiment is characterized by comprising a hollow cylindrical flexible endoscope tube 101, and a woven cable 102 arranged helically along an inner surface of the endoscope tube 101.

Inside the endoscope tube 101 are accommodated a light guide 103, an air/water supply channel 104, a suction channel 105, a treatment tool channel 106, etc., and these are protected by a protecting tube 107.

The woven cable 102 includes a plurality of electric wires 108 arranged side by side (juxtaposed) and a fiber member 109 woven in such a manner as to sew between the plurality of electric wires 108 in a side by side arrangement direction (width direction) of the plurality of the electric wires 108.

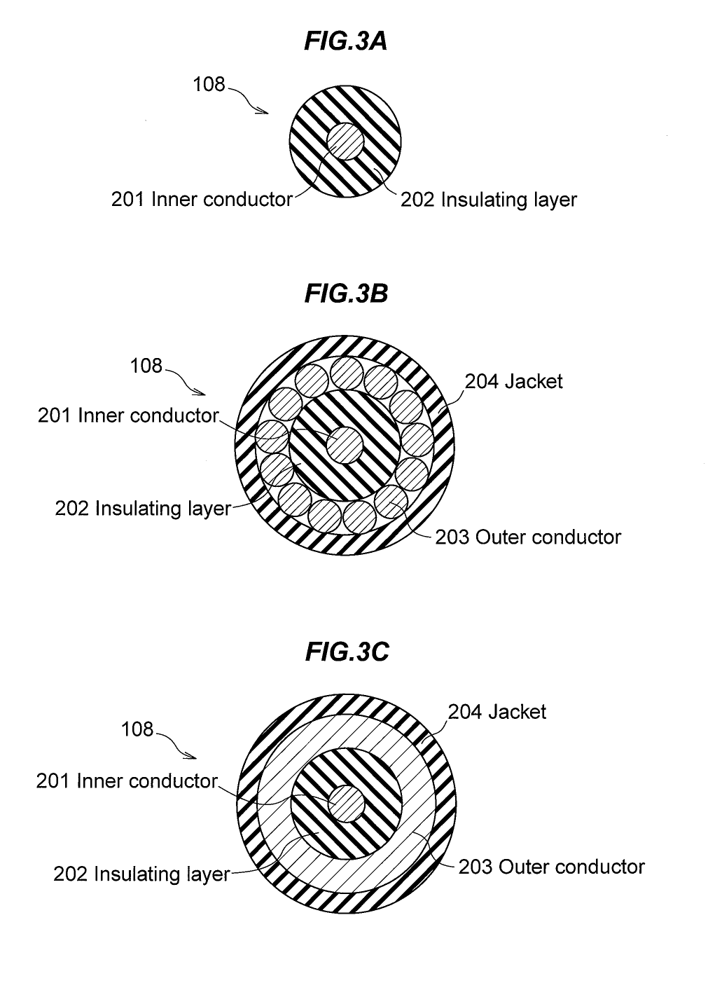

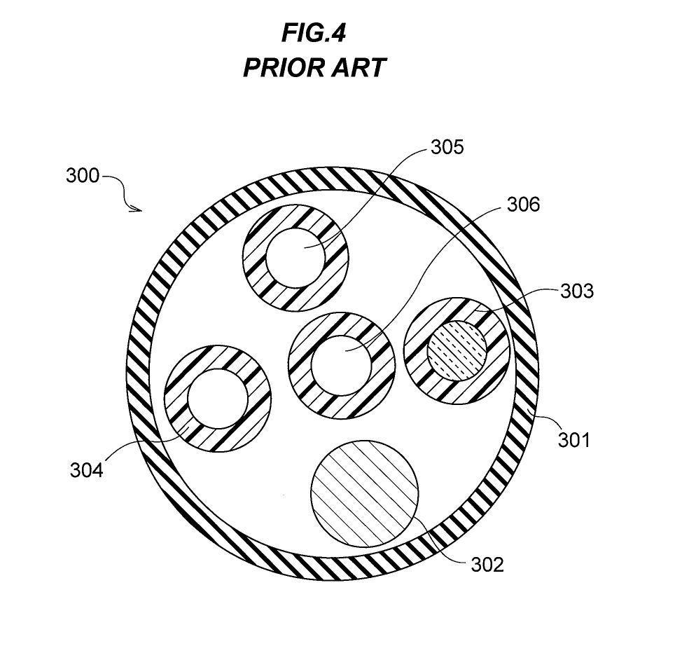

The plurality of electric wires 108 may comprise electrically insulated wires as shown in FIG. 3A or coaxial wires as shown in FIGS. 3B and 3C and these are arranged alternately or in a predetermined order to constitute the plurality of electric wires 108. Also, taking into account ensuring space saving of the woven cable 102, the plurality of electric wires 108 are preferably 0.23 mm or less in outer diameter, and are preferably 0.25 mm or less in wiring pitch.

The coaxial wire 108 includes an inner conductor 201 made of a metal wire, an insulating layer 202 formed around the inner conductor 201, outer conductors 203 formed of metal wires braided around the insulating layer 202 (as shown in FIG. 3C) or an outer conductor 203 formed of metal wires laterally wrapped around the insulating layer 202 (as shown in FIG. 3B), and a jacket 204 formed around the outer conductor(s) 203. The coaxial wire 108 is configured for electrical connection and signal transmission between the device body and the CCD image sensor.

The metal wire constituting the inner conductor 201 and the outer conductor(s) 203 is formed of copper or aluminum having excellent electrical conductivity. Also, the metal wire may be a solid wire or a stranded wire, and its surface may be plated.

The insulating layer 202 and the jacket 204 are formed of a fluoric resin such as tetrafluoroethylene perfluoroalkyl vinyl ether copolymer (PFA), tetrafluoroethylene hexafluoropropylene copolymer (FEP) or ethylene tetrafluoroethylene copolymer (ETFE), or polyethylene terephthalate (PET).

The electrically insulated wire 108 as shown in FIG. 3A includes an inner conductor 201 made of a metal wire and an electrically insulating layer 202 formed around the conductor. The electrically insulated wire 108 is configured for electrical connection between the device body and the CCD image sensor and electric power provision from the device body to the CCD image sensor.

The metal wire constituting the inner conductor 201 is formed of copper or aluminum having excellent electrical conductivity. Also, the metal wire may be a solid wire or a stranded wire, and its surface may be plated.

The insulating layer 202 is formed of a fluoric resin such as tetrafluoroethylene perfluoroalkyl vinyl ether copolymer, tetrafluoroethylene hexafluoropropylene copolymer or ethylene tetrafluoroethylene copolymer, or polyethylene terephthalate.

The fiber member 109 zigzags between the plurality of electric wires 108, from one side to the other side in a width direction (side by side arrangement direction of the plurality of electric wires 108) and from one longitudinal end to the other longitudinal end of the woven cable 102, and is woven so as to constrain and fix the plurality of electric wires 108 in a flat shape.

At this point, the fiber member 109 is preferably woven in such a manner as to sew one electric wire 108 as one unit in the middle in the width direction of the woven cable 102.

Note that the middle in the width direction of the woven cable 102 is not limited to on a central axis of the woven cable 102, but conceptually includes vicinity thereof

With these configurations, all the electric wires 108 constituting the woven cable 102 are constrained to the fiber member 109, and the plurality of electric wires 108 are arranged in such a manner as to huddle together and are aligned at a uniform wiring pitch. Therefore the width of the woven cable 102 is small and is able to contribute to the space saving of the woven cable 102, and the diametrical reduction of the endoscope cable 100.

Also, the fiber member 109 is woven in the entire longitudinal direction of the woven cable 102, but in order to facilitate connection with the device body or the CCD image sensor, the fiber member 109 woven at both longitudinal ends of the woven cable 102 may be removed.

At this point, without special work to dissolve the fiber member 109 with a solvent, the electric wires 108 and the fiber member 109 are easily separated only by pulling a tip of the fiber member 109. It is therefore possible to simplify connection with the device body or the CCD image sensor, and reduce a burden on a user.

The fiber member 109 is made of a fiber having high elongation and low initial modulus, more specifically, a polyurethane elastic fiber having a high elongation of not less than 500% and not more than 900%, an elongation recovery rate at the time of 300% elongation of not less than 90%, and an initial modulus for 300% elongation of not less than 5 cN/dtex and not more than 30 cN/dtex.

The reason for setting the elongation at not less than 500% and not more than 900% is because at less than 500%, when the woven cable 102 is bent or twisted, the fiber member 109 cannot sufficiently follow that bending or twisting.

Also, it is because at more than 900% the function of the fiber member 109 for constraining and fixing the plurality of electric wires 108 lowers.

The reason for setting the elongation recovery rate at the time of 300% elongation at not less than 90% is because at less than 90%, when the woven cable 102 is bent, the fiber member 109 is fully stretched, causing the woven cable 102 to be unlikely to return to its shape before its bending.

The reason for setting the initial modulus for 300% elongation at not less than 5 cN/dtex and not more than 30 cN/dtex is because at less than 5 cN/dtex, when the fiber member 109 is woven between the plurality of electric wires 108, the plurality of electric wires 108 cannot sufficiently be constrained with the fiber member 109, and the woven cable 102 in a good shape cannot be produced, but requires a separate subsequent step for arranging a good shape of the woven cable 102, leading to a rise in production cost.

Also, it is because at more than 30 cN/dtex, when the fiber member 109 is woven between the plurality of electric wires 108, the fiber member 109 strongly tightens the electric wires 108, being likely to cause undulation and break resulting therefrom in the electric wires 108, worsening electrical characteristics thereof.

That is, setting the initial modulus for 300% elongation at not less than 5 cN/dtex and not more than 30 cN/dtex allows the fiber member 109 to be woven between the plurality of electric wires 108 with no extra load acting on the electric wires 108.

As the polyurethane elastic fiber meeting these conditions, there is ROICA (trademark, Asahi Kasei Fibers Corporation), for example. Also, the fiber member 109 is made of preferably a monofilament from the point of view of textile strength enhancement and space saving of the woven cable 102. The use of this polyurethane elastic fiber as the fiber member 109 allows the fiber member 109 to have very thin fineness, be substantially elongated and woven between the plurality of electric wires 108.

For example, it is possible to weave the fiber member 109 having not less than 17 dtex and not more than 45 dtex by elongation to 300%. The outer diameter of the 300% elongated fiber member 109 having not less than 17 dtex and not more than 45 dtex is 0.04 mm or less.

And after weaving the fiber member 109, the plurality of electric wires 108 are arranged in such a manner as to huddle together by elongation recovery of the fiber member 109, but because the elongation of the fiber member 109 is high, no excessive force is applied to the plurality of electric wires 108.

Therefore, even when the outer diameter of the electric wires 108 is small, the elongation recovery of the fiber member 109 allows the fiber member 109 to be woven between the plurality of electric wires 108 with no stress causing a small bend in the electric wires 108, and with no undulation and break resulting therefrom being caused in the electric wires 108.

This allows for shortening the separation interval (wiring pitch) between the adjacent electric wires 108 with no extra load acting on the electric wires 108, and thereby narrowing the width of the woven cable 102, compared with the conventional width.

Further, the fiber member 109 formed of the previously described polyurethane elastic fiber is able to sufficiently elongate even after being woven between the plurality of electric wires 108. Therefore, the fiber member 109 allows the woven cable 102 to serve to stretch or compress in the width direction thereof.

This allows the woven cable 102 to sufficiently follow the bending or twisting of the endoscope cable 100. It is therefore possible to enhance the bending resistance and the twisting resistance of the endoscope cable 100.

Also, the fiber member 109 is woven preferably at a texture density of not less than 10 turns and not more than 20 turns per 1 cm in the cable longitudinal direction of the woven cable 102. The reason for weaving the fiber member 109 at a texture density of not less than 10 turns and not more than 20 turns per 1 cm in the cable longitudinal direction of the woven cable 102 is because if the texture density is less than 10 turns per 1 cm, the plurality of electric wires 108 cannot sufficiently be bundled together, while if the texture density exceeds 20 turns per 1 cm, the flexibility of the woven cable 102 is spoiled.

This woven cable 102 allows most of the load due to the bending or twisting of the endoscope cable 100 to escape to the very elastic fiber member 109. Therefore, the woven cable 102 can withstand the load due to the bending or twisting of the endoscope cable 100, and thereby prevent the break of the electric wires 108 due to the bending or twisting of the endoscope cable 100.

Further, in the woven cable 102, the plurality of electric wires 108 are made integral by the fiber member 109. Therefore, the plurality of electric wires 108 are unlikely to be separated, even if subjected to bending or twisting of the endoscope cable 100. This prevents the electric wires 108 from slipping, projecting from the woven cable 102 and being acted on by excessive load.

Also, even if the plurality of electric wires 108 are not designed to be diametrically thinned, the woven cable 102 allows for achieving space saving in comparison with the conventional electric cable 302. Therefore, no lowering in electrical characteristics and in amount of electric power provided due to the diametrical reduction of the electric wires 108 occurs. Neither terminal workability worsening nor wire breaking during handling occurs.

From the point of view of the terminal workability, because in the woven cable 102 the plurality of electric wires 108 are tied together with the fiber member 109, and the arrangement thereof is consistent in a cable longitudinal direction, the assortment of the electric wires 108 at the time of the terminal working is advantageously facilitated as well, as compared with in the conventional electric cable 302.

Further, in the woven cable 102, by forming the plurality of electric wires 108 from a coaxial wire, electromagnetic shielding can be applied thereto. Therefore, no problem as in using the flexible flat cable arises.

As described so far, the endoscope cable 100 in the present embodiment includes the woven cable 102 arranged helically inside the endoscope tube 101. It is therefore possible to achieve a further diametrical reduction as compared with a conventional diameter. In addition, the use of the stretchability in the width direction of the woven cable 102 allows substantial enhancement in bending resistance and twisting resistance in comparison with the multiconductor cable having longitudinally arranged and accommodated therein a plurality of plain weave units comprising a plurality of electric wires plain woven with a weft.

Also, in the endoscope cable 100 in the present embodiment, the woven cable 102 is arranged helically inside the endoscope tube 101. It is therefore possible to prevent entanglement between the other light guides 103, the water supply channel 104, the suction channel 105 or the treatment tool channel 106 and the woven cable 102, and thereby prevent the occurrence of breaking in the plurality of constituent electric wires 108 of the woven cable 102.

Note that the invention is not limited to the above embodiment, but various alterations may be made without departing from the spirit and scope of the invention.

For example, although in this embodiment it has been described that the woven cable 102 electrically connects together the device body and the CCD image sensor, and provides electric power from the device body to the CCD image sensor, the woven cable 102, when used for an endoscopic ultrasonograph, may electrically connect together the device body and an ultrasonic probe and provides electric power from the device body to the ultrasonic probe.

Also, although in this embodiment it has been described that the woven cable 102 is arranged helically along the inner surface of the endoscope tube 101, the woven cable 102 may be wound helically around the protecting tube 107.

Although the invention has been described with respect to the specific embodiments for complete and clear disclosure, the appended claims are not to be thus limited but are to be construed as embodying all modifications and alternative constructions that may occur to one skilled in the art which fairly fall within the basic teaching herein set forth.

What is claimed is:

1. An endoscope cable, comprising:
a hollow cylindrical flexible endoscope tube; and
a woven cable helically arranged inside the endoscope tube.

2. The endoscope cable according to claim 1, wherein the woven cable includes a plurality of electric wires arranged side by side, and a fiber member woven in such a manner as to sew between the plurality of the electric wires and in a side by side arrangement direction of the plurality of the electric wires, and the fiber member comprises a polyurethane elastic fiber.

3. The endoscope cable according to claim 2, wherein the fiber member is being elongated by weaving in such a manner as to sew between the plurality of the electric wires.

4. The endoscope cable according to claim 2, wherein the fiber member comprises a monofilament.

5. The endoscope cable according to claim 1, wherein the woven cable stretches or compresses in a side by side arrangement direction of a plurality of electric wires thereof

6. The endoscope cable according to claim 1, wherein the electric wire is 0.23 mm or less in outer diameter, and is 0.25 mm or less in wiring pitch.
